(19) United States
(12) Patent Application Publication
(10) Pub. No.: US 2025/0165660 A1
(43) Pub. Date: May 22, 2025

(54) COLLABORATIVE DESIGN SYSTEM AND METHOD FOR THERMOPHYSICAL PROPERTY GRADIENT DISTRIBUTION AND BRAIDED STRUCTURE OF CERAMIC MATRIX COMPOSITE (CMC) AND STORAGE MEDIUM

(71) Applicant: NANJING UNIVERSITY OF AERONAUTICS AND ASTRONAUTICS, Nanjing (CN)

(72) Inventors: Junkui MAO, Nanjing (CN); Zecan TU, Nanjing (CN); Yanchao CHU, Nanjing (CN); Mingdong ZHAO, Nanjing (CN); Zihao BAO, Nanjing (CN); Zhixuan REN, Nanjing (CN)

(73) Assignee: NANJING UNIVERSITY OF AERONAUTICS AND ASTRONAUTICS, Nanjing (CN)

(21) Appl. No.: 18/950,011

(22) Filed: Nov. 16, 2024

(30) Foreign Application Priority Data
Nov. 16, 2023 (CN) 202311524123.0

(51) Int. Cl. G06F 30/15

(52) U.S. Cl. CPC G06F 30/15

(57) ABSTRACT

Disclosed is a collaborative design system and method for a thermophysical property gradient distribution and a braided structure of a CMC and a storage medium. The system comprises at least one storage medium and at least one processor. The at least one processor is configured to: generate a temperature field parameter of a surface of a CMC turbine blade under a non-uniform inflow condition based on a simulation analysis platform, and extract a convective heat transfer coefficient; partition the CMC turbine blade and import an extracted fluid-solid heat transfer boundary condition into an optimization platform; perform multi-objective optimization on the thermophysical property gradient distribution of the CMC turbine blade under the non-uniform inflow condition based on an optimization simulation tool; and generate C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

### CROSS-REFERENCE TO RELATED APPLICATIONS

This application claims priority of Chinese Patent Application No. 202311524123.0, filed on Nov. 16, 2023, the entire contents of which are incorporated herein by reference.

### TECHNICAL FIELD

The present disclosure relates to the technical field of engineering thermophysics, and in particular to a collaborative design system and method for a thermophysical property gradient distribution and a braided structure of a ceramic matrix composite (CMC) and a storage medium.

### BACKGROUND

With the continuous development of aero-engine technology, and the constant improvement of various performance indicators of engines, the requirements for materials of aero-engines are also getting higher and higher. Especially with the increase in engine thrust-weight ratio, the temperature of turbine inlet gas is becoming progressively higher. In modern aero-engines, the combustion chamber temperature reaches as high as 2200K. Even with cooling techniques like blending, the temperature of gas discharged from the combustion chamber still exceeds 1600K, which has already exceeded the heat resistance limit of metal turbine blades. Therefore, the ceramic matrix composites (CMC), which possess excellent high-temperature resistance and mechanical properties, have become one of the most promising research directions.

### SUMMARY

One or more embodiments of the present disclosure provide a collaborative design system for a thermophysical property gradient distribution and a braided structure of a ceramic matrix composite (CMC). The collaborative design system may comprise: at least one storage medium including a set of instructions for storing a collaborative design of the thermophysical property gradient distribution and the braided structure of the CMC; and at least one processor in communication with the at least one storage medium. When executing the set of instructions, the at least one processor may be configured to direct the system to perform operations including: generating a temperature field parameter of a surface of a CMC turbine blade under a non-uniform inflow condition based on a simulation analysis platform, and extracting a convective heat transfer coefficient; partitioning the CMC turbine blade and importing an extracted fluid-solid heat transfer boundary condition into an optimization platform; performing multi-objective optimization on the thermophysical property gradient distribution of the CMC turbine blade under the non-uniform inflow condition based on an optimization simulation tool; and generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

One or more embodiments of the present disclosure provide a collaborative design method for a thermophysical property gradient distribution and a braided structure of a ceramic matrix composite (CMC). The collaborative design method may be implemented by at least one processor, comprising: generating a temperature field parameter of a surface of a CMC turbine blade under a non-uniform inflow condition based on a simulation analysis platform, and extracting a convective heat transfer coefficient; partitioning the CMC turbine blade and importing an extracted fluid-solid heat transfer boundary condition into an optimization platform; performing multi-objective optimization on the thermophysical property gradient distribution of the CMC turbine blade under the non-uniform inflow condition based on an optimization simulation tool; and generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

One or more embodiments of the present disclosure provide a non-transitory computer readable medium, comprising computer instructions that, when executed by at least one processor, may direct the at least one processor to perform a method comprising: generating a temperature field parameter of a surface of a CMC turbine blade under a non-uniform inflow condition based on a simulation analysis platform, and extracting a convective heat transfer coefficient; partitioning the CMC turbine blade and importing an extracted fluid-solid heat transfer boundary condition into optimization platform; performing multi-objective optimization on the thermophysical property gradient distribution of the CMC turbine blade under the non-uniform inflow condition based on an optimization simulation tool; and generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

### BRIEF DESCRIPTION OF THE DRAWINGS

FIG. 1 is a flowchart illustrating an exemplary collaborative design method for a thermophysical property gradient distribution and a braided structure of a CMC according to some embodiments of the present disclosure;

FIG. 2 is a triaxial high pressure model according to some embodiments of the present disclosure;

FIG. 3 is a model of a triaxial high pressure blade and upper and lower flange plates according to some embodiments of the present disclosure;

FIG. 4 is a fluid domain model according to some embodiments of the present disclosure; and

FIG. 5 is a schematic diagram illustrating partitions of a guide blade model according to some embodiments of the present disclosure.

FIG. 6A is a schematic diagram illustrating an exemplary experimental piece with 0% C nanotube content according to some embodiments of the present disclosure;

FIG. 6B is a schematic diagram illustrating an exemplary experimental piece with 3.75% C nanotube content according to some embodiments of the present disclosure;

FIG. 6C is a schematic diagram illustrating an exemplary experimental piece with 7.5% C nanotube content according to some embodiments of the present disclosure;

FIG. 6D is a schematic diagram illustrating an exemplary experimental piece with 11.25% C nanotube content according to some embodiments of the present disclosure; and

FIG. 6E is a schematic diagram illustrating an exemplary experimental piece with 15% C nanotube content according to some embodiments of the present disclosure.

### DETAILED DESCRIPTION

The accompanying drawings required to be used in the description of the embodiments are briefly described below. The accompanying drawings do not represent the entirety of the embodiments.

As shown in the present disclosure and in the claims, unless the context clearly suggests an exception, the words “one”, “a”, “an”, “one kind”, and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

Since the temperature of high-temperature gas discharged from a combustion chamber is non-uniform, there is a significant temperature gradient across hot end components of an aero-engine. CMCs are quite sensitive to thermal stress due to the significant difference in thermal expansion coefficients between fibers and matrixes. NASA reported that if the turbine inlet temperature exceeds 1750K, refined analysis and strict requirements are needed for the stress levels of CMC turbine components due to the fact that the internal stress caused by temperature differences has a significant impact on the strength of reinforcing fibers within the CMC.

Currently, most studies on turbine blades under a non-uniform inflow condition are based on metal blades, while there are few studies on the temperature field characteristics of CMC turbine blades. However, according to the analysis of thermophysical properties of the CMCs and the results of the temperature field characteristics obtained under a uniform inflow condition, the temperature field characteristics of the metal blades under the non-uniform inflow condition are certainly different from the temperature field characteristics of the CMC turbine blades under the non-uniform inflow condition. Moreover, because material thermal conductivities of the CMCs are related to parameters such as a material weaving pattern, a weaving angle, a fiber thermal conductivity, a matrix thermal conductivity, etc., thermophysical parameters of the CMC turbine blades have a certain designability based on the microstructure inside the material, which provides a certain space for optimizing the thermophysical gradient of the CMC turbine blades under the non-uniform inflow condition.

Because the exhaust temperature of the combustion chamber of the modern aero-engine is getting higher and higher, the exhaust temperature after blending and cooling is getting closer to an ultimate heat-resistant temperature of the CMCs. Therefore, it is necessary to explore the mechanism of heat transfer within the CMC with the braided structure under a non-uniform inflow thermal load and conduct an optimization design of the internal structure of the CMC and a thermophysical property gradient distribution of turbine blades, so as to reduce the maximum temperature and the temperature gradient of the CMC turbine blades under the non-uniform inflow thermal load.

Aiming at the need for efficient heat evacuation, the present disclosure considers that the CMC with the braided structure has heterogeneous and anisotropic characteristics, and the traditional thermal analysis method based on homogeneous metallic materials have difficulty in reflecting the effects of material structural features on internal heat transfer and temperature field distribution. Therefore, an optimization design of the internal structure of the CMC and the thermophysical property gradient distribution of the turbine blades is carried out to reduce the maximum temperature and the temperature gradients of the CMC turbine blades under the non-uniform inflow thermal load.

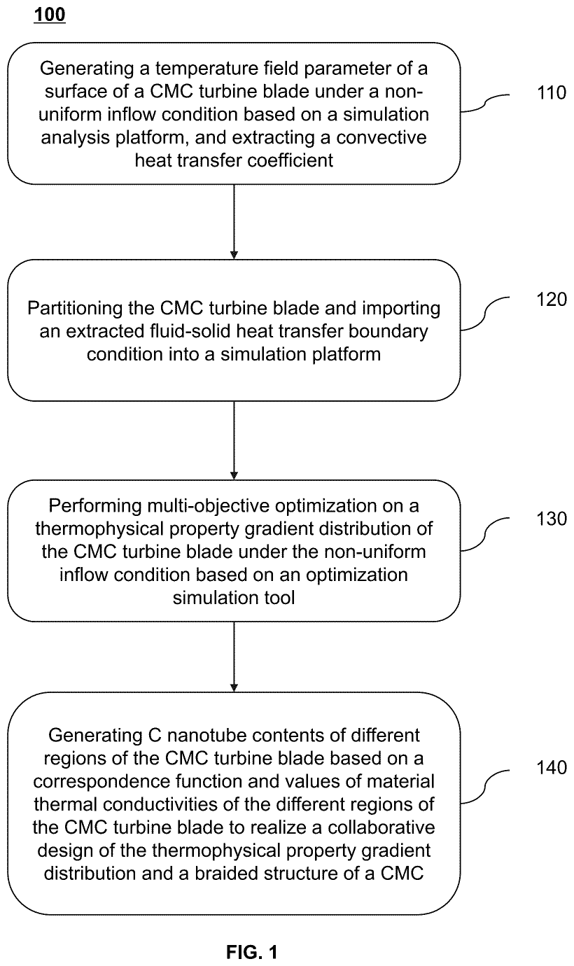

FIG. 1 is a flowchart illustrating an exemplary collaborative design method for a thermophysical property gradient distribution and a braided structure of a CMC according to some embodiments of the present disclosure. In some embodiments, a process 100 may include the following operations, as shown in FIG. 1. The process 100 may be stored in a storage medium in the form of instructions and executed by a processor.

In some embodiments, the processor may generate a temperature field parameter of a surface of a CMC turbine blade under a non-uniform inflow condition based on a simulation analysis platform, and extract a convective heat transfer coefficient; partition the CMC turbine blade and import an extracted fluid-solid heat transfer boundary condition into an optimization platform; perform multi-objective optimization on the thermophysical property gradient distribution of the CMC turbine blade under the non-uniform inflow condition based on an optimization simulation tool; and generate C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

The storage medium may be configured to store data, instructions, and/or any other information. The storage medium may include one or more storage components. Each of the one or more storage components may be an independent device or part of another device. In some embodiments, the storage medium may include a random access memory (RAM), a read-only memory (ROM), a removable memory, or the like, or any combination thereof. In some embodiments, at least one storage media may be provided.

The processor may process data and/or information obtained from other devices or system components. The processor may execute program instructions based on the data, the information, and/or processing results to perform one or more of the functions described in the application. In some embodiments, the processor may include one or more sub-processing devices (e.g., a single-core processing device or a multi-core processing device). Merely by way of example, the processor may include a central processing unit (CPU), an application-specific integrated circuit (ASIC), a microprocessor, or the like, or any combination thereof. In some embodiments, at least one processor may be provided.

In 110, a temperature field parameter of a surface of a CMC turbine blade under a non-uniform inflow condition may be generated based on a simulation analysis platform, and a convective heat transfer coefficient may be extracted.

The simulation analysis platform refers to a platform for simulation and analysis of parameters, or the like of the CMC turbine blade. For example, the simulation analysis platform may include a fluent platform, or the like. fluent is a computational fluid dynamics (CFD) software tool developed by Ansys for simulation and analysis of fluid flow, heat transfer, and related physical phenomena.

The non-uniform inflow condition refers to a condition that the velocity, direction and/or flow rate of the fluid change in space during the flow process, resulting in streamlines not being parallel to each other.

In some embodiments, the non-uniform inflow condition may be given in the form of a hot spot at a turbine gas inlet.

The hot spot at the turbine gas inlet refers to a phenomenon of an obvious circumferential and radial temperature gradient in a temperature field of a combustion chamber outlet of a gas turbine, where the temperature of a high-temperature core region is significantly higher than that of a surrounding fluid. The temperature field refers to a collection of temperatures at various points within a material system. In this case the temperature field refers to a collection of temperatures at various points at the combustion chamber outlet of the gas turbine. The combustion chamber refers to an actual space in which a fuel-air mixture is combusted to release energy. The circumferential refers to a direction of rotation around a certain central axis; and the radial refers to a direction extending outward from a central axis or a center point. The temperature gradient refers to a change rate of temperature per unit distance or per unit height.

The turbine blade is an important component of a turbine, configured to convert the energy of combustion gas into mechanical energy for driving a compressor or generator. The CMC turbine blade refers to a turbine blade made of the CMC.

In some embodiments, a material setting of the CMC turbine blade may be set in a customized manner.

The material setting of the CMC turbine blade refers to a setting of a ratio of various materials used to make the CMC turbine blade.

The customized manner refers to a personalized setting or adjustment based on actual needs. For example, the customized manner may include a user defined function (UDF), or the like. The UDF means that a user can write codes to extend functions of a software or implement specific simulation requirements.

The temperature field of the surface of the CMC turbine blade refers to a collection of temperatures at various points on the surface of the CMC turbine blade.

The temperature field parameter refers to a relevant parameter that characterizes a distribution and a variation of temperatures at various points on the surface of the CMC turbine blade. For example, the temperature field parameter may include a conductivity, a density, a specific heat, an expansion coefficient, a surface heat exchange condition, or the like. The conductivity refers to an amount of heat transferred per unit area of the CMC turbine blade per unit of time. The specific heat refers to an amount of heat required to increase or decrease the temperature of the CMC turbine blade by 1 degree per unit mass. The expansion coefficient refers to a change rate of a volume of the CMC turbine blade when the temperature changes. The surface heat exchange condition refers to a heat exchange between the surface of the CMC turbine blade and the surrounding environment.

In some embodiments, the processor may calculate the temperature field parameter of the surface of the CMC turbine blade under the non-uniform inflow condition based on the fluent platform.

The convective heat transfer coefficient refers to a parameter that characterizes a heat transfer capability between the fluid and the surface of the CMC turbine blade.

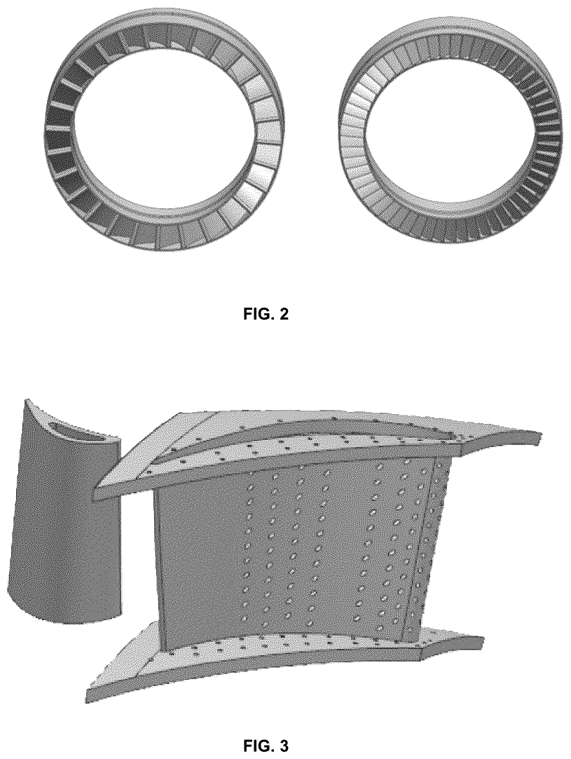

In some embodiments, the processor may customize a convective heat transfer coefficient parameter based on the fluent platform to extract the convective heat transfer coefficient. More descriptions regarding the convective heat transfer coefficient parameter may be found elsewhere in the present disclosure (e.g., related descriptions in FIGS. 2-5).

In 120, the CMC turbine blade may be partitioned and an extracted fluid-solid heat transfer boundary condition may be imported into an optimization platform.

The fluid-solid heat transfer boundary condition refers to a manner and a condition of heat transfer at an interface between the fluid and the CMC turbine blade. For example, the fluid-solid heat transfer boundary condition may include the temperature field parameter, the convective heat transfer coefficient, or the like.

The optimization platform refers to a computer platform configured to model and simulate various systems and processes. For example, the optimization platform may include a COMSOL platform, or the like. The COMSOL platform refers to a software environment for multi-physics field simulation, and is configured to help users simulate and analyze a plurality of physical phenomena and interactions thereof.

In some embodiments, the processor may partition the turbine blade in various ways and import the extracted fluid-solid heat transfer boundary condition into the optimization platform. For example, the turbine blade may be partitioned via a staff setting or a system default setting for partitioning, and the extracted fluid-solid heat transfer boundary condition may be imported into the optimization platform.

In some embodiments, the processor may partition the CMC turbine blade based on UV lines on the surface of the CMC turbine blade, save a material thermal conductivity of each region, and import the temperature field parameter of the surface of the CMC turbine blade generated by the simulation analysis platform and the convective heat transfer coefficient into the optimization platform as input parameters using an interpolation method.

In some embodiments, after the processor partitions the CMC turbine blade, the CMC turbine blade may be divided into a plurality of regions.

The UV lines refer to lines configured to connect different UV coordinate points in a UV coordinate system. The UV coordinate system is a two-dimensional coordinate system, and U and V denote the horizontal and vertical coordinates, respectively. UV coordinates are configured to represent positions on a texture image.

In some embodiments, the processor may create a three-dimensional (3D) model of the CMC turbine blade through a software (e.g., a CAD software), import the three-dimensional model of the CMC turbine blade into an appropriate modeling software (e.g., Maya), unfold the model of the turbine blade using a UV texture editor, and draw reference lines in the UV editor to obtain the UV lines.

In some embodiments, the processor may obtain a plurality of regions by separating based on the UV lines on the surface of the CMC turbine blade as the plurality of regions after the CMC turbine blade is partitioned.

The material thermal conductivity refers to an amount of heat transferred through an area of one square meter in one second when a temperature difference between two surfaces of a one-meter-thick material is 1 degree (K, ° C.) under a steady-state heat transfer condition.

The input parameters refer to relevant parameters input to the optimization platform.

The interpolation refers to a process of interpolating a continuous function on the basis of discrete data such that the continuous curve passes through all given discrete data points. The interpolation method may include nearest neighbor interpolation, linear interpolation, bilinear interpolation, trilinear interpolation, or the like.

In some embodiments, the processor may save the material thermal conductivity of each region based on partitioning results described above. The processor may take the temperature field parameter of the surface of the CMC turbine blade generated by the simulation analysis platform and the convective heat transfer coefficient as the input parameters, and generate the temperature field parameter and the convective heat transfer coefficient in a format required by the optimization platform through a numerical computation software, and then import the temperature field parameter and the convective heat transfer coefficient in the format required by the optimization platform into the optimization platform. In this content, the numerical computation software may include MATLAB, Python, Mathematica, or the like.

In some embodiments of the present disclosure, partitioning the CMC turbine blade by the UV lines ensures a more accurate partitioning; and by importing the temperature field parameter and the convective heat transfer coefficient into the optimization platform as the input parameters using the interpolation method, the more accurate thermal analysis and fluid dynamics simulation can be realized in the optimization platform, which improves the simulation accuracy of the optimization platform.

In 130, multi-objective optimization may be performed on a thermophysical property gradient distribution of the CMC turbine blade under the non-uniform inflow condition based on an optimization simulation tool.

The optimization simulation tool refers to a tool for simulating and optimizing the thermophysical property gradient distribution of the CMC turbine blade. For example, the optimization simulation tool may include a COMSOL livelink for MATLAB tool, or the like. COMSOL livelink for MATLAB is an integrated tool that integrates COMSOL Multiphysics and MATLAB, enabling users to create, edit, and run COMSOL Multiphysics models using the powerful programming and data manipulation capabilities of MATLAB.

The thermophysical property gradient distribution refers to a variation of thermophysical property parameters of the material of the CMC turbine blade in different regions. The thermophysical property parameters may include the material thermal conductivity, or the like.

The multi-objective optimization refers to a process of finding a solution that achieves the optimal balance among a plurality of objectives in a situation with a plurality of decision variables and a plurality of objective functions.

In some embodiments, the processor may perform the multi-objective optimization on the thermophysical property gradient distribution of the CMC turbine blade under the non-uniform inflow condition based on the optimization simulation tool in various ways. For example, modes of the multi-objective optimization may include multi-objective particle swarm optimization (MOPSO), an enhanced genetic algorithm (EGA), multi-objective differential evolution (MODE), or the like.

In some embodiments, the multi-objective optimization may be a non-dominated sorting genetic algorithm NSGA-II, an optimization objective may be a maximum temperature of the surface of the CMC turbine blade and a maximum temperature gradient of the CMC turbine blade, and an optimization parameter may be the material thermal conductivity of each region.

The NSGA-II is a multi-objective optimization algorithm that maintains population diversity through non-dominated sorting and crowding distance calculation, and selects superior individuals for crossover and mutation operations in each generation to ultimately yield a Pareto optimal solution set. The Pareto optimal solution set refers to a set of solutions that cannot improve one objective without deteriorating other objectives in a multi-objective optimization problem.

In some embodiments, the NSGA-II may be executed by MATLAB, where optimization initial variables and optimization objective computational solutions are obtained by MATLAB controlled COMSOL computation. More descriptions regarding specific implementations of the NSGA-II may be found elsewhere in the present disclosure (e.g., related descriptions in FIGS. 2-5).

The maximum temperature of the surface is the highest temperature of the surface of the CMC turbine blade. The lower the maximum temperature of the surface, the better the CMC turbine blade.

The maximum temperature gradient refers to a maximum value of the temperature gradient. The lower the maximum temperature gradient, the better the CMC turbine blade.

The optimization parameter refers to the material thermal conductivity of each region.

In some embodiments of the present disclosure, by using the NSGA II, the multi-objective optimization problem can be handled efficiently. By gradually improving the results through multiple iterations and selecting the relatively optimal solution in the global situation, the material thermal conductivity manifesting the most balanced and suitable maximum temperature of the surface of the CMC turbine blade and the maximum temperature gradient of the CMC turbine blade can be obtained. Meanwhile, parallel computing is supported, thereby improving computing efficiency.

In 140, C nanotube contents of different regions of the CMC turbine blade may be generated based on a correspondence function and values of material thermal conductivities of the different regions of the CMC turbine blade to realize a collaborative design of the thermophysical property gradient distribution and a braided structure of a CMC.

The correspondence function refers to a function that characterizes a correspondence between the material thermal conductivity and the C nanotube content.

In some embodiments, the correspondence function may be determined in various ways. For example, the correspondence function may be preset based on experience, or the like.

In some embodiments, the processor may obtain a material thermal conductivity of each of a plurality of experimental pieces in each direction, a C nanotube content of each of the plurality of experimental pieces being different, the material thermal conductivity being determined based on an experimental test approach; and establish the correspondence function between the material thermal conductivity and the C nanotube content.

The plurality of experimental pieces refer to objects or materials configured for testing or research. In some embodiments, the plurality of experimental pieces are 2.5D CMC pieces. The CMC pieces refer to parts made from the CMC. 2.5D is a concept between 2D and 3D, referring to introducing certain 3D effects in 2D, or using certain constraints in 3D to retain some planar features.

It should be noted that the material thermal conductivity of each of the plurality of experimental pieces in each direction refers to a macro equivalent thermal conductivity of each experimental piece in each direction. The macro equivalent thermal conductivity refers to a thermal conductivity of the CMC on a macroscopic scale. The macro equivalent thermal conductivity considers individual components of the material and thermal conductivity properties thereof.

In some embodiments, the material thermal conductivity of each of the plurality of experimental pieces in each direction may be determined based on the experimental test approach. The experimental test approach refers to an approach of testing by experimentation. For example, the experimental test approach may include a thermofluid meter method, a thermal conductivity instrument method, or the like.

The C nanotube content refers to a mass percentage or volume percentage of C nanotubes in a composite material or mixture. The C nanotubes, also referred to as buckytubes, are a type of one-dimensional quantum material having a special structure with a radial dimension being at a nanometer scale, an axial dimension being at a micrometer scale, and two ends of the tubes being essentially sealed.

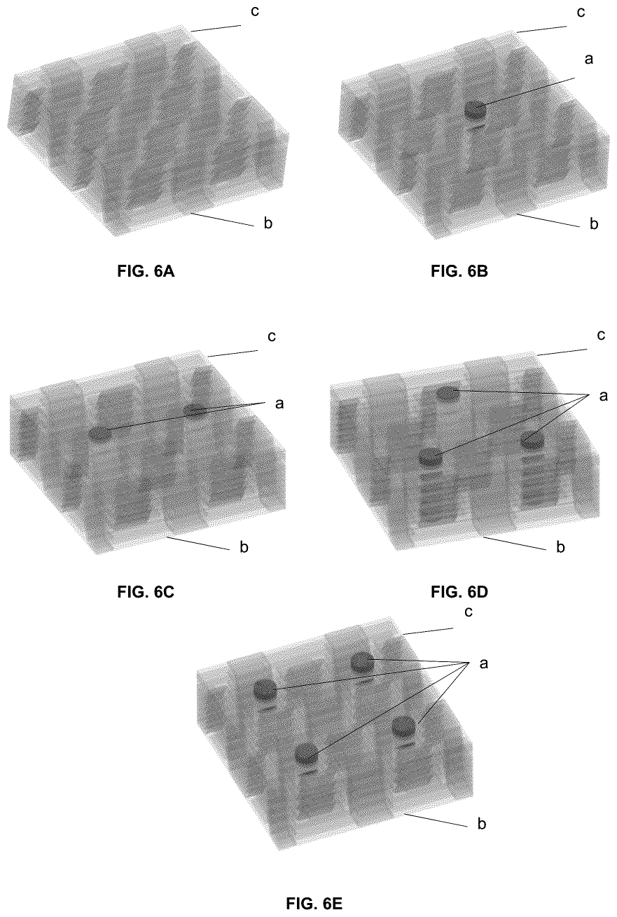

In some embodiments, in the correspondence function, the C nanotube content may include at least one of 0%, 3.75%, 7.5%, 11.25%, and 15%. It can be understood that in order to ensure that in the correspondence function, the C nanotube content includes at least one of 0%, 3.75%, 7.5%, 11.25%, and 15%, the plurality of experimental pieces may include an experimental piece with 0% C nanotube content, an experimental piece with 3.75% C nanotube content, an experimental piece with 7.5% C nanotube content, an experimental piece with 11.25% C nanotube content, and an experimental piece with 15% C nanotube content. For example, as shown in FIGS. 6A-6E, FIG. 6A is a schematic diagram illustrating an exemplary experimental piece with 0% C nanotube content according to some embodiments of the present disclosure, FIG. 6B is a schematic diagram illustrating an exemplary experimental piece with 3.75% C nanotube content according to some embodiments of the present disclosure, FIG. 6C is a schematic diagram illustrating an exemplary experimental piece with 7.5% C nanotube content according to some embodiments of the present disclosure, FIG. 6D is a schematic diagram illustrating an exemplary experimental piece with 11.25% C nanotube content according to some embodiments of the present disclosure, and FIG. 6E is a schematic diagram illustrating an exemplary experimental piece with 15% C nanotube content according to some embodiments of the present disclosure. As shown in FIGS. 6A-6E, a represents C nanotubes, b represents fibers of the CMC, and c represents a matrix of the CMC.

In some embodiments of the present disclosure, by conducting experimental testing with different C nanotube contents, the resulting correspondence function can be more accurate and have a broader coverage.

In some embodiments, after the material thermal conductivity and the corresponding C nanotube content are determined, the processor may establish a bi-directional mapping relationship between the material thermal conductivity and the C nanotube content to obtain the correspondence function. More descriptions regarding the correspondence function may be found elsewhere in the present disclosure (e.g., related descriptions in FIGS. 2-5).

In some embodiments of the present disclosure, by obtaining the material thermal conductivities of the experimental pieces corresponding to different C nanotube contents, and further establishing the correspondence function between the material thermal conductivities and the C nanotube contents, the time for subsequent determination of the C nanotube contents in the different regions of the CMC turbine blade can be effectively reduced, thereby realizing the optimization design of the thermophysical property gradient distribution and the braided structure of the CMC turbine blade.

In some embodiments of the present disclosure, the accuracy of the relevant parameters obtained can be improved by using the simulation analysis platform, the optimization platform, or the like, and time consumption can be effectively reduced. The multi-objective optimization of the thermophysical property gradient distribution of the CMC turbine blade by the optimization simulation tool can increase the running speed of the optimization process and improve reasonableness of the optimization result. With the correspondence function, the C nanotube contents corresponding to the material thermal conductivities of the different regions of the CMC turbine blade can be accurately obtained, which realizes the optimization design of the thermophysical property gradient distribution and the braided structure of the CMC turbine blade.

The collaborative design method for the thermophysical property gradient distribution and the braided structure of the CMC is described in details below with reference to embodiments. It should be noted that the platforms, data, or the like in the embodiments are only intended to illustrate the collaborative design method for the thermophysical property gradient distribution and the braided structure of the CMC, and do not limit the scope of protection of the present disclosure.

### Embodiment

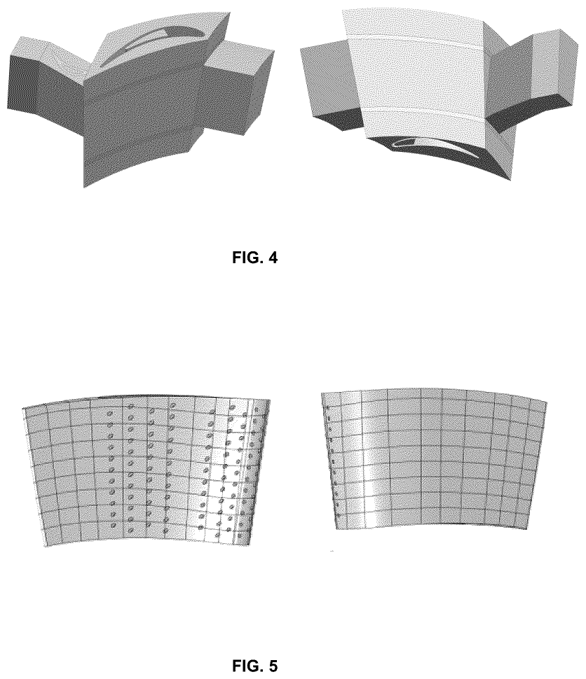

FIG. 2 is a triaxial high pressure model according to some embodiments of the present disclosure. FIG. 3 is a model of a triaxial high pressure blade and upper and lower flange plates according to some embodiments of the present disclosure. FIG. 4 is a fluid domain model according to some embodiments of the present disclosure. FIG. 5 is a schematic diagram illustrating partitions of a guide blade model according to some embodiments of the present disclosure.

The specific implementation operations of a collaborative design method for a thermophysical property gradient distribution and a braided structure of a CMC are demonstrated in the embodiment. A model used in this embodiment is shown in FIG. 2. The model is a triaxial high pressure model including guide blades, moving blades, and upper and lower flange plates. There are 28 guide blades and 57 moving blades. It should be noted that the guide blade is short for a turbine guide blade, and the guide blade may be a specific implementation of a CMC turbine blade shown in FIG. 1.

For the convenience of simulation computation, in this embodiment, the model is segmented periodically and a fluid domain model used for simulation is added. The model of the moving blades, static blades, and the upper and lower flange plates used for simulation is shown in FIG. 3. A fluid domain includes a static domain of a guide blade portion and a rotation domain of a moving blade portion. A rotation angle of a fluid domain periodic surface of the moving blade portion is 6.31578947°, and a rotation angle of a fluid domain periodic surface of the guide blade portion is 12.857142857°. The fluid domain model is shown in FIG. 4.

(1) In material setting, a material thermal conductivity of the CMC turbine blade is set to change with a blade profile through a UDF using a fluent platform. The fluid is air, and a boundary condition is calculated using a result obtained from a project experiment as a reference input.

After the computation is completed, since the subsequent optimization process is carried out in a COMSOL platform, a fluid-solid heat transfer boundary condition of a surface of a guide blade calculated in the fluent platform needs to be derived. The method of deriving a convective heat transfer coefficient of the surface of the guide blade used in this embodiment is as follows. Firstly, a convective heat transfer coefficient parameter is customized in the fluent platform, the convective heat transfer coefficient parameter being defined as shown in equation (1):

$$h = \frac{q}{( T - {T}_{0} )} \qquad ( 1 )$$

where h denotes the convective heat transfer coefficient of the surface of the guide blade, q denotes a convective heat flux, T denotes the temperature of the fluid near the guide blade, and T₀ denotes a surface temperature of the guide blade. It should be noted that since the maximum temperature of the surface of the guide blade is 1504K, the surface fluid temperature of a cavity portion of the guide blade is set to be 1600K, and the surface fluid temperature of a trailing edge portion is set to be 1400K in this embodiment. The computation in the COMSOL platform requires that the given convective heat transfer coefficients are all positive, so after the convective heat transfer coefficient of the surface of the guide blade is derived, an absolute value of the derived data is obtained.

(2) In order to reduce the computational amount, the optimization computation is performed only for a thermophysical property parameter of a solid domain guide blade model. For setting the material thermophysical properties during subsequent optimization, partitioning is required for the guide blade model before computation. The partitioning results are shown in FIG. 5. A total count of partitions of the guide blade is 163.

In the COMSOL platform, properties such as material density and thermal melting of all partitions are set to be the same as those of the CMC manufactured by Suzhou Sailifei Co., Ltd. The material thermal conductivity is set to be isotropic, and initial values of the material thermal conductivities of all partitions are set to 20 W/(K·m).

An inner surface of the guide blade and an air film hole are set as a convective heat transfer boundary, and the convective heat transfer boundary derived from the fluent platform is imported by an interpolation method.

(3) The tool used for the optimization simulation of this embodiment is the COMSOL livelink for MATLAB tool. The optimization method used is the NSGA-II. An optimization variable is the material thermal conductivity of each region of the guide blade, and an optimization objective is to reduce the maximum temperature of the surface of the guide blade and reduce the maximum temperature gradient of the guide blade. The NSGA-II is executed by MATLAB, and optimization initial variables and optimization objective computational solutions are obtained by MATLAB controlled COMSOL computation.

In this embodiment, a population size for the optimization is set to 70, a count of individual genes corresponds to the 163 partitions of the guide blade of the CMC turbine blade, an optimization range for the material thermal conductivity is 10-50 W/(K·m), and a maximum count of iterations is set to 100, with a crossover ratio of 80% and a mutation ratio of 30%.

After the optimization by the NSGA-II, the maximum temperature of the surface of the guide blade is 1457.41K, and the maximum temperature gradient is 158,402.52 K/m. The maximum temperature of the surface of the guide blade is reduced by 45.4318 K compared to 1502.85K before optimization. The maximum temperature gradient is reduced by 87024.1006 K/m compared to 245426.62 K/m before optimization. The maximum temperature gradient after optimization is reduced by 35.458% compared to the maximum temperature gradient before optimization.

(4) Experimental testing of a 2.5D CMC component with C nanotube contents of 0%, 3.75%, 7.5%, 11.25%, and 15% to determine the material thermal conductivity in each direction is conducted, and a bi-directional mapping relationship between the material thermal conductivity of the CMC component and the C nanotube content of the CMC component is established to determine the correspondence function. The C nanotube contents of different regions of the guide blade are determined based on the material thermal conductivity of each region of the guide blade obtained from the optimization, and the optimization design for the thermophysical property gradient distribution and the braided structure of the guide blade is realized.

In some embodiments of the present disclosure, the optimization design of the internal structure of the CMC and the thermophysical property gradient distribution of the turbine blade is carried out based on the designability of the internal microscopic structure of the material aiming at the demand for highly efficient heat evacuation of the aero-engine CMC turbine blade. The maximum temperature of the surface of the CMC turbine blade is slightly reduced, and the material temperature gradient of the CMC turbine blade is substantially reduced. Finally, the experiments are carried out to test the material thermal conductivity of the CMC doped with different C nanotube contents in each direction, and the correspondence function between the material thermal conductivity of the CMC and the C nanotube content of the CMC is established. The C nanotube contents of the different regions of the CMC turbine blade are determined based on the results of the material thermal conductivities of the different regions of the CMC turbine blade obtained from the optimization, such that the optimization design of the thermophysical property gradient distribution and the braided structure of the CMC turbine blade is realized, and the maximum temperature and the temperature gradient of the CMC turbine blade are reduced.

One or more embodiments of the present disclosure provide a collaborative design system for a thermophysical property gradient distribution and a braided structure of a CMC. The collaborative design system may comprise at least one storage medium including a set of instructions for storing a collaborative design of the thermophysical property gradient distribution and the braided structure of the CMC; and at least one processor in communication with the at least one storage medium. When executing the set of instructions, the at least one processor may be configured to direct the system to perform operations including: generating a temperature field parameter of a surface of a CMC turbine blade under a non-uniform inflow condition based on a simulation analysis platform, and extracting a convective heat transfer coefficient; partitioning the CMC turbine blade and importing an extracted fluid-solid heat transfer boundary condition into an optimization platform; performing multi-objective optimization on the thermophysical property gradient distribution of the CMC turbine blade under the non-uniform inflow condition based on an optimization simulation tool; and generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

In some embodiments, the at least one processor may be further configured to: obtain a material thermal conductivity of each of a plurality of experimental pieces in each direction, a C nanotube content of each of the plurality of experimental pieces being different, the material thermal conductivity being determined based on an experimental test approach; and establish the correspondence function between the material thermal conductivity and the C nanotube content.

In some embodiments, the non-uniform inflow condition may be given in the form of a hot spot at a turbine gas inlet, and a material setting of the CMC turbine blade may be set in a customized manner.

In some embodiments, the at least one processor may be configured to: partition the CMC turbine blade based on UV lines on the surface of the CMC turbine blade, save a material thermal conductivity of each region, and import the temperature field parameter of the surface of the CMC turbine blade generated by the simulation analysis platform and the convective heat transfer coefficient into the optimization platform as input parameters using an interpolation method.

In some embodiments, the multi-objective optimization may be the NSGA-II, an optimization objective may be a maximum temperature of the surface of the CMC turbine blade and a maximum temperature gradient of the CMC turbine blade, and an optimization parameter may be the material thermal conductivity of each region.

In some embodiments, in the correspondence function, the C nanotube content may include at least one of 0%, 3.75%, 7.5%, 11.25%, and 15%. The plurality of experimental pieces may be 2.5D CMC pieces.

One or more embodiments of the present disclosure provide a non-transitory computer readable medium, comprising computer instructions that, when executed by at least one processor, may direct the at least one processor to perform a method comprising: generating a temperature field parameter of a surface of a CMC turbine blade under a non-uniform inflow condition based on a simulation analysis platform, and extracting a convective heat transfer coefficient; partitioning the CMC turbine blade and importing an extracted fluid-solid heat transfer boundary condition into an optimization platform; performing multi-objective optimization on the thermophysical property gradient distribution of the CMC turbine blade under the non-uniform inflow condition based on an optimization simulation tool; and generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

When the operations performed in the embodiments of the present disclosure are described in terms of steps, the order of the steps are all interchangeable if not otherwise indicated, and some steps are omissible. Other steps may be included in the operations.

The embodiments of the present disclosure are for the purpose of exemplification and illustration only and do not limit the scope of application of the present disclosure. To a person skilled in the art, various corrections and changes that may be made under the guidance of the present disclosure remain within the scope of the present disclosure.

Some features, structures, or characteristics of one or more embodiments of the present disclosure may be appropriately combined.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

What is claimed is:

1. A collaborative design system for a thermophysical property gradient distribution and a braided structure of a ceramic matrix composite (CMC), comprising:
at least one storage medium including a set of instructions for storing a collaborative design of the thermophysical property gradient distribution and the braided structure of the CMC; and
at least one processor in communication with the at least one storage medium, wherein when executing the set of instructions, the at least one processor is configured to direct the system to perform operations including:
generating a temperature field parameter of a surface of a CMC turbine blade under a non-uniform inflow condition based on a simulation analysis platform, and extracting a convective heat transfer coefficient;
partitioning the CMC turbine blade and importing an extracted fluid-solid heat transfer boundary condition into an optimization platform;
performing multi-objective optimization on the thermophysical property gradient distribution of the CMC turbine blade under the non-uniform inflow condition based on an optimization simulation tool; and
generating C nanotube contents in different regions of the CMC turbine blade, based on a correspondence function and values of material thermal conductivities of the different regions of the CMC turbine blade, to realize the collaborative design between the thermophysical property gradient distribution and the braided structure of the CMC.

2. The collaborative design system of claim 1, wherein the at least one processor is further configured to:
obtain a material thermal conductivity of each of a plurality of experimental pieces in each direction, a C nanotube content of each of the plurality of experimental pieces being different, the material thermal conductivity being determined based on an experimental test approach; and
establish the correspondence function between the material thermal conductivity and the C nanotube content.

3. The collaborative design system of claim 1, wherein:
the non-uniform inflow condition is given in the form of a hot spot at a turbine gas inlet, and a material setting of the CMC turbine blade is set in a customized manner.

4. The collaborative design system of claim 1, wherein the at least one processor is configured to:
partition the CMC turbine blade based on UV lines on the surface of the CMC turbine blade, save a material thermal conductivity of each region, and import the temperature field parameter of the surface of the CMC turbine blade generated by the simulation analysis platform and the convective heat transfer coefficient into the optimization platform as input parameters using an interpolation method.

5. The collaborative design system of claim 4, wherein:
the multi-objective optimization is a non-dominated sorting genetic algorithm NSGA-II, an optimization objective is a maximum temperature of the surface of the CMC turbine blade and a maximum temperature gradient of the CMC turbine blade, and an optimization parameter is the material thermal conductivity of each region.

6. The collaborative design system of claim 2, wherein:
in the correspondence function, the C nanotube content includes at least one of 0%, 3.75%, 7.5%, 11.25%, and 15%, and the plurality of experimental pieces are 2.5D CMC pieces.

7. A collaborative design method for a thermophysical property gradient distribution and a braided structure of a ceramic matrix composite (CMC), implemented by at least one processor, comprising:
generating a temperature field parameter of a surface of a CMC turbine blade under a non-uniform inflow condition based on a simulation analysis platform, and extracting a convective heat transfer coefficient;
partitioning the CMC turbine blade and importing an extracted fluid-solid heat transfer boundary condition into an optimization platform;
performing multi-objective optimization on the thermophysical property gradient distribution of the CMC turbine blade under the non-uniform inflow condition based on an optimization simulation tool; and
generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.

8. The collaborative design method of claim 7, wherein determining the correspondence function includes:
obtaining a material thermal conductivity of each of a plurality of experimental pieces in each direction, a C nanotube content of each of the plurality of experimental pieces being different, the material thermal conductivity being determined based on an experimental test approach; and
establishing the correspondence function between the material thermal conductivity and the C nanotube content.

9. The collaborative design method of claim 7, wherein:
the non-uniform inflow condition is given in the form of a hot spot at a turbine gas inlet, and a material setting of the CMC turbine blade is set in a customized manner.

10. The collaborative design method of claim 7, wherein the partitioning the CMC turbine blade and importing an extracted fluid-solid heat transfer boundary condition into an optimization platform includes:
partitioning the CMC turbine blade based on UV lines on the surface of the CMC turbine blade, saving a material thermal conductivity of each region, and importing the temperature field parameter of the surface of the CMC turbine blade generated by the simulation analysis platform and the convective heat transfer coefficient into the optimization platform as input parameters using an interpolation method.

11. The collaborative design method of claim 10, wherein:
the multi-objective optimization is a non-dominated sorting genetic algorithm NSGA-II, an optimization objective is a maximum temperature of the surface of the CMC turbine blade and a maximum temperature gradient of the CMC turbine blade, and an optimization parameter is the material thermal conductivity of each region.

12. The collaborative design method of claim 8, wherein:
in the correspondence function, the C nanotube content includes at least one of 0%, 3.75%, 7.5%, 11.25%, and 15%, and the plurality of experimental pieces are 2.5D CMC pieces.

13. A non-transitory computer readable medium, comprising computer instructions that, when executed by at least one processor, direct the at least one processor to perform a method comprising:
generating a temperature field parameter of a surface of a CMC turbine blade under a non-uniform inflow condition based on a simulation analysis platform, and extracting a convective heat transfer coefficient;
partitioning the CMC turbine blade and importing an extracted fluid-solid heat transfer boundary condition into an optimization platform;
performing multi-objective optimization on the thermophysical property gradient distribution of the CMC turbine blade under the non-uniform inflow condition based on an optimization simulation tool; and
generating C nanotube contents of different regions of the CMC turbine blade based on a correspondence function and values of material thermal conductivities of the different regions of the CMC turbine blade to realize the collaborative design of the thermophysical property gradient distribution and the braided structure of the CMC.
